(19) United States
(12) Patent Application Publication
(10) Pub. No.: US 2016/0113711 A1
(43) Pub. Date: Apr. 28, 2016

(54) ABLATION CATHETER AND METHOD OF FORMING A CIRCULAR LESION

(71) Applicant: Oscor Inc., Palm Harbor, FL (US)

(72) Inventors: Thomas P. Osypka, Palm Harbor, FL (US); Timothy A. Searfoss, New Port Richey, FL (US)

(73) Assignee: Oscor Inc., Palm Harbor, FL (US)

(21) Appl. No.: 14/920,300

(22) Filed: Oct. 22, 2015

Related U.S. Application Data
(60) Provisional application No. 62/067,135, filed on Oct. 22, 2014.

(51) Int. Cl. A61B 18/14

(52) U.S. Cl. CPC A61B 18/1492; A61B 2018/1407; A61B 2018/1253

(57) ABSTRACT

An ablation device is disclosed for forming a generally circular lesion on the interior wall of a blood vessel, which includes an elongated catheter deployable from a steerable guiding sheath and having a distal end portion adapted and configured for movement into a generally circular open looped condition when deployed from the guiding sheath, wherein the distal end portion of the catheter includes at least one flexible electrode that extends along the distal end portion of the catheter without interruption, so that when the distal end portion of the catheter is deployed from the guiding sheath into a generally circular open looped condition, the flexible electrode conforms to a circular configuration.

### CROSS-REFERENCE TO RELATED APPLICATIONS

The subject invention claims the benefit of priority to U.S. Provisional Patent Application Ser. No. 62/067,135, filed Oct. 22, 2014, the disclosure of which is herein incorporated by reference in its entirety.

### BACKGROUND OF THE INVENTION

1. Field of the Invention

The subject invention is directed to an intravascular medical device, and more particularly, to an ablation catheter for forming a generally circular lesion on the interior wall of a blood vessel or on cardiac tissue, and to a method of treating atrial fibrillation and/or renal hypertension.

2. Description of Related Art

Ablation systems using radio frequency (RF) energy are well known, and are often used to treat unwanted electrical conductivity in the human body. For example, ablation is used in the treatment of atrial fibrillation, one of the most common cardiac arrhythmias. Ablation of atrial fibrillation can be accomplished with different techniques; the most established approach is via radio frequency ablation around the pulmonary veins, which are the veins that bring oxygenated blood from the lungs back to the upper chambers or atria, in the left side of the heart.

Renal sympathetic denervation (RSDN), often shortened to renal denervation (RDN), is a minimally invasive, endovascular catheter based procedure using radiofrequency ablation aimed at treating resistant hypertension (high blood pressure). By applying radio frequency pulses to the renal arteries, the nerves in the vascular wall (adventitia layer) can be denuded of nerve endings. This causes reduction of renal sympathetic afferent and efferent activity and blood pressure can be decreased.

Ablation systems commonly include a steerable ablation catheter having electrically conductive electrodes surrounding the distal section of the catheter, and the catheter is connected to an energy source, such as an RF ablation generator. The catheter often includes a handle, which allows the deflection of the distal catheter section, so the catheter can be introduced and steered towards its target location in a blood vessel. The catheters typically have electrically conductive ring electrodes made form Pt/Ir, Ag, stainless steel or other conductive materials. The electrode configurations are designed and optimized for ablation procedures, where the target areas are small spots or areas.

To increase the size of the ablation areas, some systems add saline solution at the distal tip location of the catheter, so that during the ablation procedure the tip is cooled. Thus, so the target area can be ablated longer and deeper. For pulmonary vein ablation, which is commonly used to treat atrial fibrillation, the desirable is not only to ablate one or more small spots inside the left atrial wall, but rather it is desired to ablate a complete continuous circumferential line inside of the pulmonary vein.

To achieve this, prior art ablation systems have been provided with a deployable tip portion 10 shaped in a circular loop, as shown for example in FIGS. 1 and 2. In these prior art devices, which normally range in size from 5 F to 7 F in diameter, a plurality of cylindrical electrodes 12 are spaced along the circumference of the distal looped portion 10, which is deployable from a guiding sheath 14. The electrodes 12 can be activated either in a unipolar or bipolar multiplexed configuration to ablate a plurality of target tissue locations on the inside surface of the pulmonary vein. However, such catheter designs typically result in only partially successful ablation procedures, since some spots between the electrodes do not get ablated during the procedure.

Another disadvantage of such ablation systems is that during the procedure the ablation energy is applied to the entirety of each cylindrical electrode 12. Thus, not only are the target tissue areas heated by the electrodes, the surrounding blood is also heated, creating possible negative side effects such as coagulation. To overcome this problem and to perform complete circumferential ablation in the pulmonary vein, cryo-ablation systems are used as an alternative, an example of which is shown in FIG. 3.

The prior art cryo-ablation device shown in FIG. 3 has an elongated catheter 12 with a distally located balloon 16 which, when deployed from a sheath 14 is inflated and cooled down to −50 degrees F., and in that inflated and cooled state it used to freeze and ablate the tissue of the wall of the pulmonary vein in a circumferential manner. However, this prior art device has the disadvantage that the spherical shape of the balloon does necessarily match the circumferential surface of the pulmonary vein. As a result, missing spots will often remain on the pulmonary vein wall. Another disadvantage of the cryo-ablation system is that the catheter must be insulated to prevent it from cooling other areas of the vessel or heart. In addition, the size of cryo-ablation catheter is more than double that of current RF ablation devices, ranging typically from 12 F to 14 F in catheter diameter.

It is therefore desirable to provide an ablation system, which on the one hand can ablate a target area within a pulmonary vein in a complete uninterrupted circular manner, while on the other hand can conform flexibly to the shape of the inside wall of the pulmonary vein, and at the same time be smaller in size than current RF ablation catheters.

In addition it is desirable to have an ablation system which only treats and heats the target tissue area, while preventing heat increase of non-target areas, such as the surrounding blood.

### SUMMARY OF THE INVENTION

The subject invention is directed to a new and useful ablation device for forming a generally circular lesion on the interior wall of a blood vessel while flexibly conforming to the shape of the blood vessel and heating only the target tissue, without unnecessarily heating the surrounding blood.

The device includes an elongated catheter that is deployable from an elongated guiding sheath, which is preferably a steerable sheath. The device has a distal end portion that is adapted and configured for movement into a generally circular open looped condition when it is deployed from the end of the guiding sheath. Preferably, the ablation catheter has a shaft size of about between 1.0 and 2.0 mm and an operative length of about between 145 and 165 cm. The distal end portion of the catheter preferably has a loop size of about between 15 and 20 mm, depending upon the intended use or ablation procedure being performed therewith.

The open looped distal end portion of the ablation catheter includes at least one flexible electrode that extends along a substantial part of the distal end portion of the catheter without interruption. Thus, when the distal end portion of the catheter is deployed from the guiding sheath into a generally circular open looped condition, the flexible electrode conforms to a circular configuration. The circular configuration and size of the looped portion of the catheter closely matches the shape or contour of the inner wall of the blood vessel into which it is guided and deployed. Preferably, the at least one flexible electrode is formed at least partially from shape memory alloy.

In one embodiment of the subject invention, the catheter is configured for unipolar ablation and thus includes a single flexible electrode. In another embodiment of the subject invention, the catheter is configured for bipolar ablation and includes a pair of flexible electrodes. In this instance, the ablation catheter includes two laterally spaced apart flexible electrodes, each wrapped partially around the circumference of the distal end portion of the catheter to define an outer peripheral gap therebetween.

In yet another embodiment of the subject invention, the ablation catheter includes three laterally spaced apart flexible electrodes, each wrapped partially around the circumference of the distal end portion of the catheter, with gaps therebetween. In this instance, the third electrode may be selectively used alone for unipolar ablation or in combination with one of the other electrodes for bipolar ablation.

The subject invention is also directed to an ablation system for forming a generally circular lesion on the interior wall of a blood vessel. The system includes a steerable guiding sheath having an elongated body with opposed proximal and distal ends, and an elongated ablation catheter deployable from the distal end of the steerable guiding sheath. Preferably, the steerable guiding sheath has an actuation assembly operatively associated with a handle at the proximal end of the sheath for steering the distal end of the heath to a target location within a blood vessel.

The ablation catheter has a distal end portion adapted and configured for movement into a generally circular open looped condition when deployed from the distal end of the steerable guiding sheath. The distal end portion of the ablation catheter includes at least one flexible electrode that extends along the distal end portion of the ablation catheter without interruption, so that when the distal end portion of the ablation catheter is deployed from the distal end of the steerable guiding sheath into a generally circular open looped condition, the flexible electrode conforms to a circular configuration that matches the shape of the blood vessel.

The subject invention is also directed to a treatment method, which includes the steps of: guiding an intravascular sheath to a target location within a blood vessel; deploying an ablation catheter loop from a distal end of the sheath so that an electrode extending continuously along an outer periphery of the catheter loop is in substantial contact with an interior wall of the blood vessel; and then energizing the electrode to form a generally circular lesion on the interior wall of the blood vessel through RF ablation. In accordance with the invention, the generally circular lesion can be formed through either unipolar RF ablation or bipolar RF ablation depending upon the electrode configuration.

In one embodiment of the subject invention, the treatment method includes the step of guiding the intravascular sheath to a target location in the pulmonary vein of a patient to treat atrial fibrillation. In another embodiment of the subject invention, the treatment method includes the step of guiding the intravascular sheath to a target location in the renal vein of a patient to treat hypertension.

These and other features of the subject invention and the manner in which it is manufactured and employed for treatment will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the ablation system of the subject invention appertains will readily understand how to make and use the subject invention without undue experimentation, preferred embodiments thereof will be described in detail herein below with reference to certain figures, wherein:

FIG. 1 is an illustration of a prior art ablation system deployed through a guiding sheath, which includes a distal loop with a plurality of spaced apart electrodes for performing circular RF ablation within a blood vessel;

FIG. 2 is an illustration of another prior art ablation system having a deployable distal loop with spaced apart electrodes for performing circular ablation within a blood vessel;

FIG. 3 is an illustration of yet another prior art ablation system deployed through a guiding sheath, which includes a balloon assembly that is inflated and cooled within a blood vessel to perform cryo-ablation;

FIG. 4 is a perspective view of the distal end portion of an elongated guiding sheath with the ablation loop of the subject invention retracted therein prior to deployment within a blood vessel;

FIG. 5 is a perspective view of the ablation loop of the subject invention deployed from the distal end portion of the guiding sheath shown in FIG. 4, wherein the ablation loop is configured for bipolar circular ablation;

FIG. 6 is a side elevational view of the deployed ablation loop shown in FIG. 5;

FIG. 7 is a cross-sectional view taken along line 7-7 of FIG. 6, showing the wiring in the interior of the catheter loop;

FIG. 8 is an elevational view of a handle assembly for steering the guiding sheath to traverse the vasculature of a patient to facilitate deployment of the ablation loop of the subject invention at a target location within a blood vessel;

FIG. 9 is a perspective view of another embodiment of the ablation loop of the subject invention deployed from the distal end of a guiding sheath, wherein the ablation loop is configured for unipolar circular ablation;

FIG. 10 is a side elevational view of the deployed ablation loop shown in FIG. 9;

FIG. 11 is a cross-sectional view taken along line 11-11 of FIG. 10, showing the interior wiring of the ablation loop; and

FIG. 12 is a perspective view of yet another embodiment of the ablation loop of the subject invention deployed from the distal end of a guiding sheath, wherein the ablation loop includes three circular electrodes configured for unipolar or bipolar circular ablation.

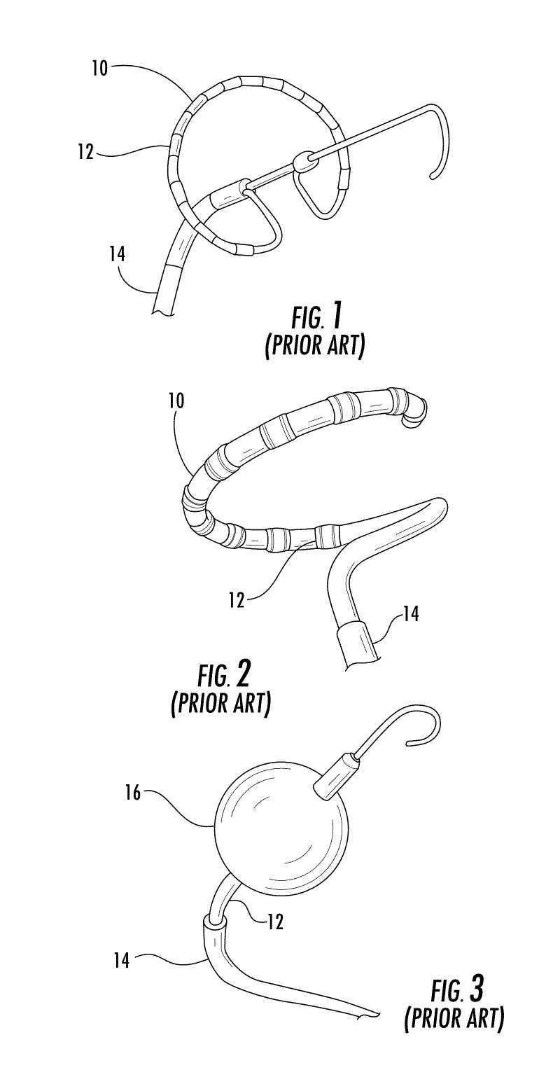

### DETAILED DESCRIPTION OF THE INVENTION

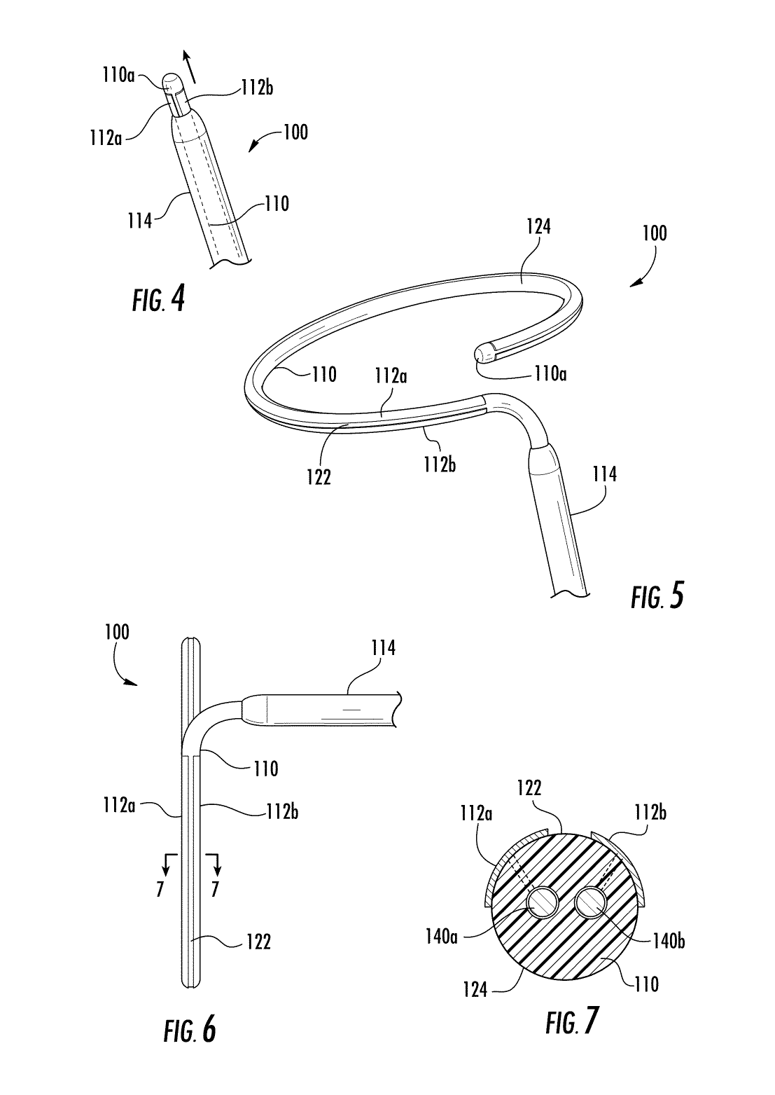

Referring now to the drawings wherein like reference numerals identify similar structural features of the disclosed device, there is illustrated in FIGS. 4-7, a closed loop circular ablation system 100 designed to ablate, in a complete uninterrupted circular manner, the inside of a human artery or vein, such as the pulmonary vein entering the left atrium of the human heart, to treat atrial fibrillation (or other treatments such as the inside of the renal artery to treat hypertension), utilizing radio frequency (RF) energy.

The ablation system 100 is a single use device that includes an elongated flexible ablation catheter 110. The distal end portion of catheter 110 is mounted for movement relative to an elongated guiding sheath 114 between a retracted position stowed within the distal end of the sheath 114, as shown in FIG. 4, and a deployed position extended from the distal end of the sheath 114, as shown in FIGS. 5 and 6. In the deployed position of FIGS. 5 and 6, the distal end portion of catheter 110 has an open looped or generally circular shape that closely matches the shape of the blood vessel within which it is deployed.

Preferably, the ablation catheter 110 has a shaft size of about between 1.0 and 2.0 mm and an operative length of about between 145 and 165 cm. The distal end portion of the catheter 110 preferably has a loop size of about between 15 and 20 mm. These dimensions can vary depending upon the ablation procedure. The distal-most end of the catheter loop has an atraumatic tip 110a.

To achieve bipolar ablation of the inner wall of a blood vessel, the distal end portion of the catheter 110 includes a pair of spaced apart continuous electrode loops 112a and 112b which are attached along a substantial part of the length of the distal end portion. The electrodes 112a, 112b are separated from one another along their lengths by an outer peripheral gap 122 and a larger inner peripheral gap 124. The outer gap 122 allows for the bipolar ablation of tissue in a complete circular pattern without interruption. The inner gap 124 ensures that blood in the vessel is not unnecessarily heated during an ablation procedure.

The electrodes 112a, 112b are flexible and preferably formed from a shape memory metal alloy such a Nickel and Titanium alloy, whereby the shape memory alloy has a normally unstressed circular loop configuration when it is deployed from the sheath 114, as illustrated in FIG. 5. Alternatively, the polymeric material from which the distal end portion of the catheter 110 is formed may be molded into a normally looped configuration, whereby the electrodes 112a and 112b are formed from a more compliant conductive material that does not control the configuration or shape of the catheter loop when it is deployed.

It is also envisioned that the catheter 110 could include a central core component that has pre-formed circular shape to control the deployed configuration of the catheter loop. In such an instance, the material forming the catheter body and the material forming the electrode(s) would be more compliant than the shape defining core component.

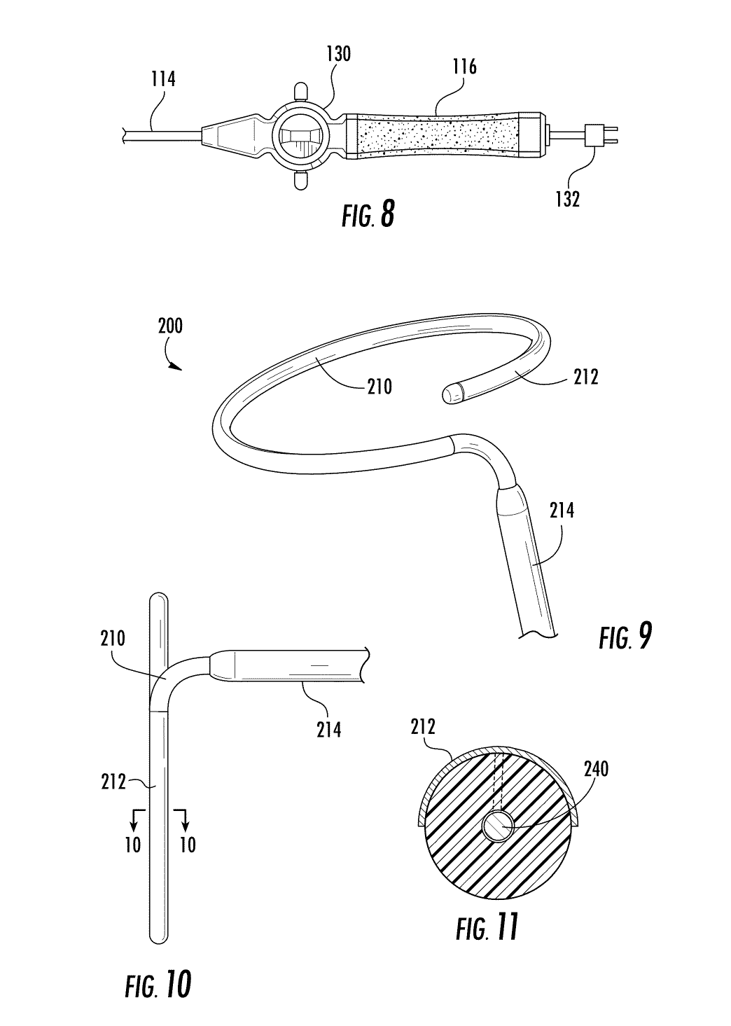

The peripherally spaced and paired electrode loops 112a, 112b are designed to extend or otherwise wrap individually around less than about 50% of the circumferential diameter or outer peripheral surface of the distal portion of the catheter body 110, as best seen in FIG. 7. This allows the RF ablation energy to be focused on the area of interest while in tissue contact, as opposed to heating up the surrounding blood, as would occur if the electrode extended about the entire periphery of the distal portion of the catheter.

Referring to FIG. 8, a handle assembly 116 is operatively associated with a proximal end portion of the guiding sheath 114 to facilitate the guided introduction of the sheath 114 through the vasculature of a patient to a target area for performing RF ablation. More particularly, the handle assembly 116 includes a steering mechanism 130 for manually controlling bi-directional or uni-directional movement of the guiding sheath 114. The steering mechanism 130 enables the distal end portion of the sheath 114 to be deflected or rotated so as to be guided by precise navigation throughout the human vessels or heart to a target location to conduct ablation. A steerable guiding sheath of this type is disclosed in commonly assigned U.S. Patent Application Publication No. 2015/0057610, which is incorporated herein by reference in its entirety.

The guiding sheath handle assembly 116 includes an electrical connector 132 for connecting the ablation catheter 110 to an RF ablation generator (not shown). The connector 132 communicates with the bipolar electrodes 112a, 112b associated with the looped distal end portion of the cannula 110, through electrical conductors 140a, 140b and associated wiring, as illustrated for example in FIG. 7.

Referring now to FIGS. 8-10, there is illustrated another embodiment of the ablation system of the subject invention, designated generally by reference numeral 200. Ablation system 200 includes an elongated catheter 210 that is deployable from a steerable sheath 214, and which is adapted and configured for unipolar ablation, as opposed to bipolar ablation. That is, the looped distal end portion of the ablation catheter 210 includes a single continuous flexible electrode 212, provided continuously along the length of the distal catheter loop. The single electrode 212 is preferably formed from a shape memory alloy that has a normally unstressed generally circular configuration that acts to define the shape of the distal loop portion of catheter 210 when it is deployed from the sheath. Alternative shaping mechanisms are envisioned here as explained above. The electrode 212 is wrapped around about 50% of the outer periphery of the distal looped portion of catheter 210, as best seen in FIG. 11. Thus the inner peripheral area of the distal loop portion of catheter 210 will not heat any blood flowing therethrough.

In this embodiment, a single electrical conductor 240 extends through the catheter 210 to deliver RF energy to the flexible electrode 212 for ablation, as shown in FIG. 10. Unipolar ablation is commonly performed by having the patient lying on an indifferent electrode, representing ground. In contrast, for bipolar ablation the second electrode represents the ground polarity.

In use, referring to FIGS. 4 and 5 for purposes of illustration and not limitation, the intravascular sheath 114 is guided to a target location within a blood vessel of a patient, typically by radioscopic vision, electrical mapping or a similar technique. In the case of treating atrial fibrillation, the sheath 114 is guided to a target area within the pulmonary vein. In the case of treating renal hypertension, the sheath 114 is guided to a target area within the renal vein.

Once the distal end of guiding sheath 114 has been properly positioned at a target location in a blood vessel, the distal end portion of the ablation catheter 110 is deployed from the stowed position of FIG. 4 to assume the curved loop configuration of FIG. 5. This occurs under the influence of the shape memory alloy from which the flexible electrodes 112a, 112b are formed, for example.

At such a time, the shaped electrodes 112a, 112b are in substantial contact with an interior wall of the blood vessel. Thereupon, the electrodes 112a, 112b are energized by the surgeon so as to form a generally circular lesion on the interior wall of the blood vessel through bipolar RF ablation, without unnecessarily increasing the temperature of surrounding tissue or blood. This same method can be performed with the unipolar ablation catheter 200.

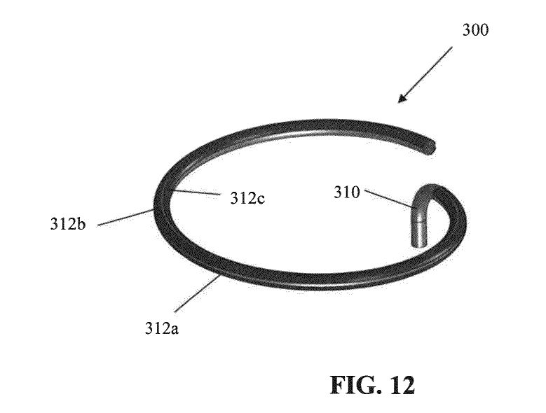

Referring now to FIG. 12, there is illustrated another embodiment of the ablation system of the subject invention designated generally by reference numeral 300, wherein the ablation catheter 310 is deployable from a steerable sheath and includes three laterally spaced apart flexible electrodes 312a, 312b and 312c, each wrapped partially around the circumference of the distal end portion of the catheter 310, with gaps therebetween. More particularly, there is a narrow gap between the first and second electrodes 312a and 312b, a second narrow gap between the second and third electrodes 312b and 312c, and a third larger gap along the inner periphery of the distal end portion of the catheter 310 between the first and third electrodes 312a and 312c.

In this embodiment of the invention, appropriate wiring and switches are provided so that the third electrode 312c may be selectively used by the surgeon either alone to perform unipolar RF ablation of the vessel wall or in combination with one of the other electrodes 312a and 312b for bipolar RF ablation of the vessel wall. When the third electrode 312c is used in this manner, either alone or in combination with one of the other two electrodes 312a or 312b, the surrounding blood will not be unnecessarily heated and the device will not block blood flow through the vessel during the procedure.

While the subject invention has been shown and described with reference to preferred embodiments, those skilled in the art will readily appreciate that various changes and/or modifications may be made thereto without departing from the spirit and scope of the subject invention as defined by the appended claims.

What is claimed is:

1. An ablation device for forming a generally circular lesion, comprising:
an elongated catheter deployable from a guiding sheath and having a distal end portion adapted and configured for movement into a generally circular open looped condition when deployed from the guiding sheath, wherein the distal end portion of the catheter includes at least one flexible electrode that extends along the distal end portion of the catheter without interruption, so that when the distal end portion of the catheter is deployed from the guiding sheath into a generally circular open looped condition, the flexible electrode conforms to a circular configuration.

2. An ablation device for forming a generally circular lesion as recited in claim 1, wherein the catheter is configured for unipolar ablation and includes at least a single flexible electrode.

3. An ablation device for forming a generally circular lesion as recited in claim 1, wherein the catheter is configured for bipolar ablation and includes at least a pair of flexible electrodes.

4. An ablation device for forming a generally circular lesion as recited in claim 3, wherein the catheter includes at least two spaced apart flexible electrodes, each wrapped partially around the circumference of the distal end portion of the catheter.

5. An ablation device for forming a generally circular lesion as recited in claim 1, wherein an electrical assembly is operatively associated with the at least one flexible electrode for delivering electrical energy thereto through the catheter.

6. An ablation device for forming a generally circular lesion as recited in claim 1, wherein the at least one flexible electrode is formed at least partially from shape memory alloy.

7. An ablation device for forming a generally circular lesion as recited in claim 1, wherein the catheter has a shaft size of about between 1 and 2 mm and an operative length of about between 145 and 165 cm.

8. An ablation device for forming a generally circular lesion as recited in claim 1, wherein the distal end portion of the catheter has a loop size of about between 15 and 20 mm.

9. An ablation system for forming a generally circular lesion, comprising:
a) a steerable guiding sheath having an elongated body with opposed proximal and distal ends;
b) an elongated ablation catheter deployable from the distal end of the steerable guiding sheath and having a distal end portion adapted and configured for movement into a generally circular open looped condition when deployed from the distal end of the guiding sheath, wherein the distal end portion of the ablation catheter includes at least one flexible electrode that extends along the distal end portion of the ablation catheter without interruption, so that when the distal end portion of the ablation catheter is deployed from the distal end of the steerable guiding sheath into a generally circular open looped condition, the at least one flexible electrode conforms to a circular configuration.

10. An ablation system for forming a generally circular lesion as recited in claim 9, wherein the ablation catheter is configured for unipolar ablation and includes at least a single flexible electrode.

11. An ablation system for forming a generally circular lesion as recited in claim 9, wherein the ablation catheter is configured for bipolar ablation and includes at least a pair of flexible electrodes.

12. An ablation system for forming a generally circular lesion as recited in claim 9, wherein the steerable guiding sheath has an actuation assembly operatively associated with the proximal end thereof for manually steering the distal end of the guiding sheath to a target location within a blood vessel.

13. An ablation system for forming a generally circular lesion as recited in claim 9, wherein an electrical wiring assembly is provided within the ablation catheter for delivering electrical energy to the at least one flexible electrode.

14. An ablation system for forming a generally circular lesion as recited in claim 9, wherein the at least one flexible electrode of the ablation catheter is formed at least partially from shape memory alloy.

15. An ablation system for forming a generally circular lesion as recited in claim 9, wherein the ablation catheter has a shaft size of about between 1 and 2 mm and an operative length of about between 145 and 165 cm.

16. An ablation device for forming a generally circular lesion as recited in claim 1, wherein the distal end portion of the ablation catheter has a loop size of about between 15 and 20 mm.

17. A treatment method, comprising the steps of:
a) guiding an intravascular sheath to a target location within a blood vessel;
b) deploying an ablation catheter loop from a distal end of the intravascular sheath so that an electrode extending continuously along an outer periphery of the catheter loop is in substantial contact with an interior wall of the blood vessel; and
c) energizing the electrode to form a generally circular lesion on the interior wall of the blood vessel.

18. A treatment method according to claim 17, including the step of guiding the intravascular sheath to a target location in the pulmonary vein of a patient to treat atrial fibrillation.

19. A treatment method according to claim 17, including the step of guiding the intravascular sheath to a target location in the renal vein of a patient to treat hypertension.

20. A treatment method according to claim 17, wherein a generally circular lesion is formed through either unipolar RF ablation or bipolar RF ablation.
